(19) United States
(12) Patent Application Publication
(10) Pub. No.: US 2010/0075314 A1
(43) Pub. Date: Mar. 25, 2010

(54) CHEMICALLY-MODIFIED GOLD NANOPARTICLES AND METHODS FOR USE IN DETECTING TARGET MOLECULES

(76) Inventors: Viswanadham Garimella, Vernon Hills, IL (US); Thomas F. Holzman, Libertyville, IL (US); Yijia P. Bao, Vernon Hills, IL (US)

Correspondence Address: GREGORY T. PLETTA;Nanosphere, Inc., 4088 Commerical Avenue, Northbrook, IL, 60062-1829, US

(21) Appl. No.: 12/472,273

(22) Filed: May 26, 2009

Related U.S. Application Data
(60) Provisional application No. 61/055,875, filed on May 23, 2008.

(51) Int. Cl. C12Q 1/68; C07J 41/00; C07F 1/12

(52) U.S. Cl. 435/6; 540/5; 540/3

(57) ABSTRACT

The invention provides stable bioconjugate-nanoparticle probes which are useful for detecting nucleic acids and other target analytes, e.g., proteins, and methods of preparing those probes.

### CROSS-REFERENCE TO RELATED APPLICATIONS

This application claims the benefit of the filing date of U.S. Ser. No. 61/055,875, filed on May 23, 2008, the disclosure of which is incorporated by reference herein in its entirety.

### FIELD OF THE INVENTION

The invention relates to stable bioconjugate-nanoparticle probes which are useful for detecting nucleic acids and other target analytes. The invention also relates to methods for preparing bioconjugate-nanoparticle probes, to methods of detecting target analytes using the probes, and to kits comprising the probes.

### BACKGROUND

The development of methods for detecting and sequencing nucleic acids is critical to the diagnosis of genetic, bacterial, and viral diseases. See Mansfield et al., Molecular and Cellular Probes, 9:145 (1995). A major challenge for those working in the area of DNA detection involves the development of methods that do not rely on polymerase chain reaction (PCR) or comparable target amplification systems. Besides added cost, methods based on PCR require additional instrumentation, thermocycling, and reagents that are not ideal for point-of-care or field use. Another restrictive requirement of most DNA detection systems, regardless of their need for PCR, is a thermal stringency wash to achieve desired analyte selectivity. In 1997, oligonucleotide-modified Au nanoparticles were introduced as colorimetric DNA detection probes (Elghanian et al. Science, 277:1078 (1997)).

DNA detection methods that employ gold nanoparticle probes, modified with oligonucleotides, to indicate the presence of a particular DNA are described in PCT/US00/17507, which is incorporated by reference herein in its entirety. Typically, oligonucleotides having sequences complementary to the nucleic acid to be detected are attached to a nanoparticle. The nanoparticle conjugate hybridized to the nucleic acid results in a detectable change resulting from the hybridization of the oligonucleotide on the nanoparticle to the nucleic acid target in solution. In order to attach the oligonucleotide to the nanoparticle, the oligonucleotide, the nanoparticle or both, are functionalized. These methods are known in the art and include, for instance, the functionalization of oligonucleotides with alkanethiols at their 3′-termini or 5′-termini. Such functionalized nucleotides readily attach to gold nanoparticles.

In contrast with other probes, such as fluorophores or radioactive labels, nanoparticles can be used in variety of detection formats that take advantage of their size dependent scattering, catalytic properties, absorption characteristics, and Raman-enhancing properties to develop systems that are substantially more sensitive and selective than their molecular fluorophore counterparts. Moreover, gold particles that have been heavily functionalized with oligonucleotides exhibit extraordinarily sharp melting profiles that translate into higher target selectivities (Storhoff et al., J. Am. Chem. Soc., 120:1959 (1998); Reynolds et al., J. Am. Chem. Soc., 122:3795 (2000); Taton et al., Science, 289:1757 (2000)). Despite the extraordinary sensitivity and selectivity of these systems, all rely on thermal stringency washes to differentiate target strands from ones with mismatches.

Colloidal gold-protein probes have found wide applications in immunocytochemistry. These probes have been prepared by adsorbing the antibodies onto the gold surface from an aqueous solution under carefully defined conditions. The complexes produced in this manner are functional but suffer from several drawbacks, e.g., some of the protein desorbs on standing, liberating antibody into solution that competes with adsorbed antibodies for the antigen target; the activity is low since the amount adsorbed is low and some of the antibody denatures on adsorption; and the protein-coated particles are prone to self aggregation (agglomeration), especially in solutions of high ionic strength. An alternative means for preparing nanoparticle-protein probes has been described in U.S. Pat. No. 5,521,289. Typically, this procedure involves reduction of a gold salt in an organic solvent containing a triarylphosphine or mercapto-alkyl derivative bearing a reactive substituent, X, to give small nanoparticles (50 to 70 gold atoms) carrying X substituents on linkers bound to the surface through Au—P or Au—S bonds. Subsequently the colloidal solution is treated with a protein bearing a substituent Y that reacts with X to link the protein covalently to the nanoparticle. Work with these nanoparticles is limited by the poor water solubility of many proteins, which limits the range of protein-nanoparticle conjugates that can be utilized effectively. Also, since there are only a few gold atoms at the surface of these particles, the number of “capture” strands that can be bound to the surface of a given particle is very low.

A variety of methods have been developed for assembling metal and semiconductor colloids into nanomaterials. These methods have focused on the use of covalent linker molecules that possess functionalities at opposing ends with chemical affinities for the colloids of interest. One of the most successful approaches to date (Brust et al., Adv. Mater., 7:795 (1995)) involves the use of gold colloids and well-established thiol adsorption chemistry (Bain & Whitesides, Angew. Chem. Int. Ed. Engl., 28:506 (1989) and Dubois & Nuzzo, Annu. Rev. Phys. Chem., 43: 437 (1992). In this approach, linear alkanedithiols are used as the particle linker molecules. The thiol groups at each end of the linker molecule covalently attach themselves to the colloidal particles to form aggregate structures. The drawbacks of this method are that the process is difficult to control and the assemblies are formed irreversibly. Methods for systematically controlling the assembly process are needed if the materials properties of these structures are to be exploited fully.

### SUMMARY OF THE INVENTION

The invention provides stable bioconjugate-nanoparticle probes which are useful for detecting nucleic acids and other target analytes, and methods of preparing those probes. In one embodiment, thiol linked methylene, ethylene or propylene glycol-biotin, e.g., polyethylene (PEG)-biotin, oligomers are prepared and then attached to nanoparticles, such as gold nanoparticles, yielding nanoparticle probes for ultra sensitive target detection. As used herein, “biotin” includes derivatives thereof that bind to avidin or streptavidin. In one embodiment, 3′-biotin labeled glycol derived oligomers are linked to gold nanoparticles via a 5′-epiendrosterone disulfide linkage. The presence of more than one thiol per oligomer likely increases the stability of the biotin labeled oligomer containing nanoparticles. In one embodiment, the 3′-biotin labeled glycol derived oligomer loaded particles are prepared or isolated in the presence of a molecule, e.g., a protein such as a phosphoprotein, e.g., casein, or serum albumin, or other proteins which can bind to and stabilize gold nanoparticles and do not substantially interfere with the assay of the invention, in an amount that increases reactivity and/or reduces non-specific binding, for instance, to surfaces. The 3′-biotin labeled glycol derived oligomer linked gold nanoparticle probes of the invention are more sensitive than nanoparticle probes prepared by other methods. A useful probe is one which is stable, does not aggregate on storage at 4° C., does not demonstrate significant non-specific binding to a surface, e.g., during storage or during target detection, and/or has specificity and sensitivity, e.g., a probe that selectively binds to the target, e.g., target antigen, which may be detected at a concentration of about 1 to 500 (or any integer in between 1 and 500) fg/mL, or about 0.1 to 100 (or any integer in between 1 and 100) pg/mL, in an assay format. As described hereinbelow, the probes of the invention met those criteria. Moreover, biotin labeled glycol derived oligomers, e.g., 3 PEGmers per each molecule, with or without DNA fillers on the surface, were loaded in about 24 hours. The data suggests that biotin loaded particles that are co-loaded with casein show higher reactivity and, as the casein concentration is increased in the co-loading and/or blocking step, the reactivity of the probe also is increased.

In one embodiment, the combination of the thiol linked PEG-biotin modified probe co-loaded with casein onto the nanoparticle increases the stability of the nanoparticles in solution, particularly for colloidal gold nanoparticles in high salt solutions, e.g., conditions including but not limited to 0.8 M sodium chloride or magnesium chloride. The use of non-nucleic acid polymers in preparing nanoparticle conjugates is advantageous for nucleic acid and protein detection because non-specific binding interference between analytes and nanoparticle conjugate probes may be reduced.

In one embodiment, the invention provides a compound which includes a steroid cyclic disulfide anchor for a gold nanoparticle surface, linked to a first glycol-based backbone (e.g., polyethylene glycol) via a phosphate linkage, which is linked to a second glycol-based backbone (e.g., triethylene glycol) via a phosphate linkage, which in turn is linked to a biotin label. In one embodiment, the compound includes: L-PO₂—O((-T-O—PO₂—O)ₘ—Zₙ)_{q}-biotin, wherein L represents a steroid with a cyclic disulfide functional group or any mono, di or multi sulfur linked aliphatic or aromatic compound; T represents thymidine but may be any nucleobase, e.g., any nucleoside, or non-nucleoside compound, e.g., one that stabilizes a gold surface; each Z independently represents a polyglycol which may be the same or different; wherein n ranges from 3 to 20; wherein q ranges from 1 to 20; wherein m is 0 or 1; and wherein each polyglycol independently comprises from 1 to 60 carbon atoms and includes from 1 to 30 non-peroxide —O— (formula (I)). In one embodiment, the compound includes: L-PO₂—O((-T-O—PO₂—O)ₘ—Zₙ)_{q}-biotin, wherein L represents a steroid with a cyclic disulfide functional group; T represents thymidine but may be any nucleobase, e.g., any nucleoside, or non-nucleoside compound, e.g., one that stabilizes a gold surface; each Z independently represents a polyethylene or triethylene glycol which may be the same or different, or a combination thereof; wherein n ranges from 3 to 20; wherein q ranges from 1 to 20; wherein m is 0 or 1; and wherein each Z independently comprises from 2 to 60 carbon atoms and includes from 1 to 30 non-peroxide —O— (formula (II)). In one embodiment, the compound includes: L-PO₂—O((-T-O—PO₂—O)ₘ—Zₙ)_{q}-biotin, wherein L represents a steroid with a cyclic disulfide functional group or any mono, di or multi sulfur linked aliphatic or aromatic compound; T represents thymidine but may be any nucleobase, e.g., any nucleoside, or non-nucleoside compound, e.g., one that stabilizes a gold surface; each Z independently represents an aliphatic, aromatic, cyclic, acyclic, anionic or cationic linker having no more than 500 atoms, which may be the same or different; wherein n ranges from 3 to 20; wherein q ranges from 1 to 20; wherein m is 0 or 1; and wherein the linker does not substantially interfere with target binding or result in significant non-specific binding to surfaces (formula (III)). In one embodiment, Z can be a polymer (e.g., polyethylene glycol or polymethylene), —C₁-C₁₀-alkyl-, —COO—, —CH₂(CH₂)ᵥCOO—, —OCO—, R¹N(CH₂)ᵥ, —O—C(CH₂)ᵥ—, —O—(CH₂)ᵥ—O—, wherein v is 0-30 and R¹ is H or is G(CH₂)ᵥ, wherein G is —CH₃, —CHCH₃, —COOH, —CO₂(CH₂)ᵥCH₃, —OH, or —CH₂OH.

In one embodiment, the invention provides nanoparticles having the compound of the invention. In one embodiment, the nanparticles are coupled to a compound of formula (I): L-PO₂—O((-T-O—PO₂—O)ₘ—Zₙ)_{q}-biotin, wherein L represents a steroid with a cyclic disulfide functional group; wherein T represents thymidine; wherein each Z independently represents a polyglycol which may be the same or different; wherein n ranges from 3 to 20; wherein q ranges from 1 to 20; wherein m is 0 or 1; and wherein each polyglycol independently comprises from 1 to 60 carbon atoms and includes from 1 to 30 non-peroxide —O—, wherein the coupling is via thiol bonds between the nanoparticle and the steroid with a cyclic functional group. In one embodiment, the compound coupled to the nanoparticles includes a compound of formula (I) wherein m is 0, q is 1, n is 4, and Z is (C₁₂O₆)₃ or C₆O₃ or any combination thereof. In one embodiment, the compound coupled to the nanoparticles includes a compound of formula (I) wherein m is 1, Z is (C₁₂O₆), n is 1, and q is 3. In one embodiment, the nanoparticles comprise gold.

Further provided is a method of preparing nanoparticles comprising the compound of formula (I), and an isolated preparation of those nanoparticles. In one embodiment, the preparation is isolated in the presence of a protein that stabilizes the preparation, reduces non-specific binding, reduces agglomeration, or any combination thereof. In one embodiment, the preparation includes about 0.005% to about 0.5% of a phosphoprotein, e.g., casein.

Also provided are methods of using the biotin probe containing nanoparticles to detect an analyte. In one embodiment, the method includes providing a support having a ligand specific for the analyte attached thereto (a capture moiety); contacting the support with a sample suspected of having the analyte so as to yield a mixture; contacting the mixture with an amount of a preparation of biotin probe containing nanoparticles, an avidin labeled molecule, and a biotinylated ligand specific for the analyte; and detecting the presence or amount of the gold nanoparticles bound to the support, thereby detecting the presence or amount of the analyte in the sample. In one embodiment, the preparation includes a protein that stabilizes the preparation, reduces non-specific binding, reduces agglomeration, or any combination thereof. The presence or amount of that protein may increase signal intensity.

### BRIEF DESCRIPTION OF THE FIGURES

FIG. 1. Schematic of nanoparticle biotin containing probes with and without DNA fillers.

FIGS. 2A-B. Schematic of target detection using biotin linked probes. A) General antibody-based assay. B) Antibody-based assay to detect troponin (cTnI).

FIG. 3. Exemplary nanoparticle biotin containing probe of the invention.

FIG. 4. Graph of signal for probes of the invention stored at 4° C. (red) or 45° C. (blue) for 48 hours. 1=No casein blocking; 2=0.01% casein blocking; 3=0.05% casein blocking; 4=0.1% casein blocking; and 5=control.

FIG. 5A. Array layout. Anti-troponin antibody 8I-7, 3=5 μg/μL, 4=2 μg/μL, 5=1 μg/μL, 6=500 pg/mL, 7=200 pg/mL, 8=100 pg/mL, 9=40 pg/mL, 10=20 pg/mL, 11=10 pg/mL, 12=2 pg/mL. All probes were loaded using 5′-PEG-PEG-PEG-biotin 3′ oligomers.

FIG. 5B. Results from the slide with the array layout shown in FIG. 5A with and without blocking agents. Arrays 1-5, streptavidin (20 ng/100 μL) added before probe (top row), and arrays 6-10, no streptavidin, only buffer and probe (lower row). Array 1, Epi-(PEG)-3-biotin; array 2, Epi-(PEG)-3-biotin, 0.1% BSA blocking for 1 hour; array 3, Epi-(PEG)-3-biotin, 0.01% casein blocking for 1 hour; array 4, Epi-(PEG)-3-biotin, 0.05% casein blocking for 1 hour; array 5, Epi-(PEG)-3-biotin, 0.1% casein blocking for 1 hour; array 6, Epi-(PEG)-3-biotin; array 7, Epi-(PEG)-3-biotin, BSA blocking for 1 hour; array 8, Epi-(PEG)-3-biotin, 0.01% casein blocking for 1 hour; array 9, Epi-(PEG)-3-biotin, 0.05% casein blocking for 1 hour; array 10, Epi-(PEG)-3-biotin, 0.1% casein blocking for 1 hour.

FIG. 6A. Array layout. Anti-troponin antibody 8I-7, 3=5 μg/μL, 4=2 μg/μL, 5=1 μg/μL, 6=500 pg/mL, 7=200 pg/mL, 8=100 pg/mL, 9=40 pg/mL, 10=20 pg/mL, 11=10 pg/mL, 12=2 pg/mL. Probes were loaded using 5′-PEG-PEG-PEG-biotin 3′ oligomers and 0.1% casein capping.

FIG. 6B. Results from the slide with the array layout shown in FIG. 6A. Arrays 1 and 6, 2 pg/mL, target; arrays 2 and 7, 1 pg/mL, target; arrays 3 and 8, 0.5 pg/mL, target; arrays 4 and 9, 0.2 pg/mL target; arrays 5 and 10, control.

FIG. 7. Graph of signal intensity (red) for different target concentrations (blue) (2000, 1000, 500, 200 or 1 fg/μL).

FIG. 8A. Array layout. Anti-troponin antibody 81-1, 3=5 μg/μL, 4=2 μg/μL, 5=1 μg/μL, 6=500 pg/mL, 7=200 pg/mL, 8=100 pg/mL, 9=40 pg/mL, 10=20 pg/mL, 11=10 pg/mL, 12=2 pg/mL.

FIG. 8B. Results from the slide with the array layout shown in FIG. 8A. Array 1, Epi-(PEG)-3-biotin stored at 4° C.; array 2, Epi-(dPEG)-3-biotin stored at 4° C., 0.01% casein blocking; array 3, Epi-(PEG)-3-biotin stored at 4° C., 0.05% casein blocking; array 4, Epi-(PEG)-3-biotin stored at 4° C., 0.1% casein blocking; array 5, no probe; array 6, Epi-(PEG)-3-biotin (50 pM) stored at 45° C.; array 7, Epi-(PEG)-3-biotin (50 pM) stored at 45° C., 0.01% casein blocking; array 8, Epi-(PEG)-3-biotin (50 pM) stored at 45° C., 0.05% casein blocking; array 9, Epi-(PEG)-3-biotin (50 pM) stored at 45° C., 0.1% casein blocking; well 10, no probe.

FIG. 9. Graph of signal intensity for probes stored at 4° C. (blue) or at 45° C. (red). 1, no casein; 2, 0.01% casein; 3, 0.05% casein; 4, 0.1% casein; 5, control (all reagents except probe).

FIG. 10. Results for a hydrogel slide with 3′ amine linked PEG-biotin (0, 4 pM, 8 pM, 16 pM, 32 pM, 62.5 pM, 125 pM, or 250 pM).

FIGS. 11A-B. Graph of signal intensity for various concentrations of probe in a streptavidin sandwich assay.

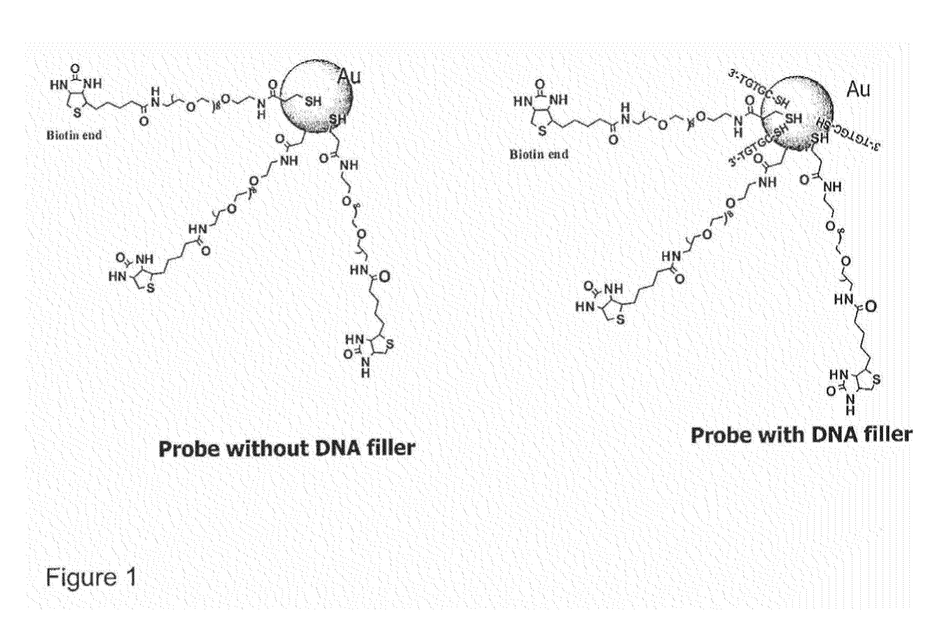

### DETAILED DESCRIPTION OF THE INVENTION

### Definitions

“Analyte” or “target analyte” is a substance to be detected in a test physiological sample using the present invention. The analyte can be any substance, e.g., a protein, or a set of related proteins, e.g., troponin isoforms and metabolites thereof.

“Capture moiety” is a specific binding member, capable of binding the analyte, which moiety may be in solution or directly or indirectly attached to a substrate. One example of a capture moiety includes an antibody bound to a support either through covalent attachment or by adsorption onto the support surface.

The term “ligand” refers to any organic compound for which a receptor or other binding molecule naturally exists or can be prepared. The term ligand also includes ligand analogs, which are modified ligands, usually an organic radical or analyte analog, usually of a molecular weight greater than 100, which can compete with the analogous ligand for a receptor, the modification providing means to join the ligand analog to another molecule. The ligand analog usually differs from the ligand by more than replacement of a hydrogen with a bond which links the ligand analog to another molecule, e.g., a label, but need not. The ligand analog can bind to the receptor in a manner similar to the ligand. The analog could be, for example, an antibody directed against the idiotype of an antibody to the ligand. For instance, a capture antibody may have a label that binds another molecule, e.g., the antibody is linked to biotin and strapetavidin is coated onto a substrate.

The term “receptor” or “antiligand” refers to any compound or composition capable of recognizing a particular spatial and polar organization of a molecule, e.g., epitopic or determinant site. Illustrative receptors include naturally occurring receptors, e.g., thyroxine binding globulin, antibodies, enzymes, Fab fragments, lectins, nucleic acids, avidin, protein A, barstar, complement component Clq, and the like. Avidin is intended to include egg white avidin and biotin binding proteins from other sources, such as streptavidin.

The term “antibody” refers to an immunoglobulin which specifically binds to and is thereby defined as complementary with a particular spatial and polar organization of another molecule, including recombinant antibodies such as chimeric antibodies and humanized antibodies. The antibody can be monoclonal or polyclonal and can be prepared by techniques that are well known in the art such as immunization of a host and collection of sera (polyclonal) or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)₂, Fab′, and the like. In addition, aggregates, polymers, and conjugates of immunoglobulins or their fragments can be used where appropriate so long as binding affinity for a particular molecule is maintained.

As used herein, the term “substrate” refers to any surface capable of having capture probes bound thereto. Such surfaces include, but are not limited to, glass, metal, plastic, or materials coated with a functional group designed for binding of capture probes or analytes. Substrates also may be referred to as slides.

### Exemplary Compounds of the Invention and Nanoparticles Including the Compounds

The invention provides biotin polymer containing nanoparticles and methods for preparing those particles. The polymers serve as “spacer” molecules between the surface of the nanoparticle and the biotin recognition moiety. These nanoparticle probes comprising polymers and a recognition element are useful for biomolecule detection (e.g. nucleic acid sequence or protein), detecting protein-ligand interactions, separation of a target oligonucleotide sequence from a population of sequences, or other methods as described previously, for instance, in PCT/US01/10071, U.S. Pat. No. 6,361,944, and U.S. Pat. No. 7,253,277, which are incorporated by reference in their entirety.

In order to attach the biotin polymer to the nanoparticle, the polymer, the nanoparticle or both, are functionalized. These methods are known in the art and include, for instance, the functionalization of the polymer with thiol linkers at their 3′-termini or 5′-termini. Such functionalized molecules readily attach to gold nanoparticles. See, for example, U.S. Pat. No. 7,186,814, incorporated herein by reference in its entirety.

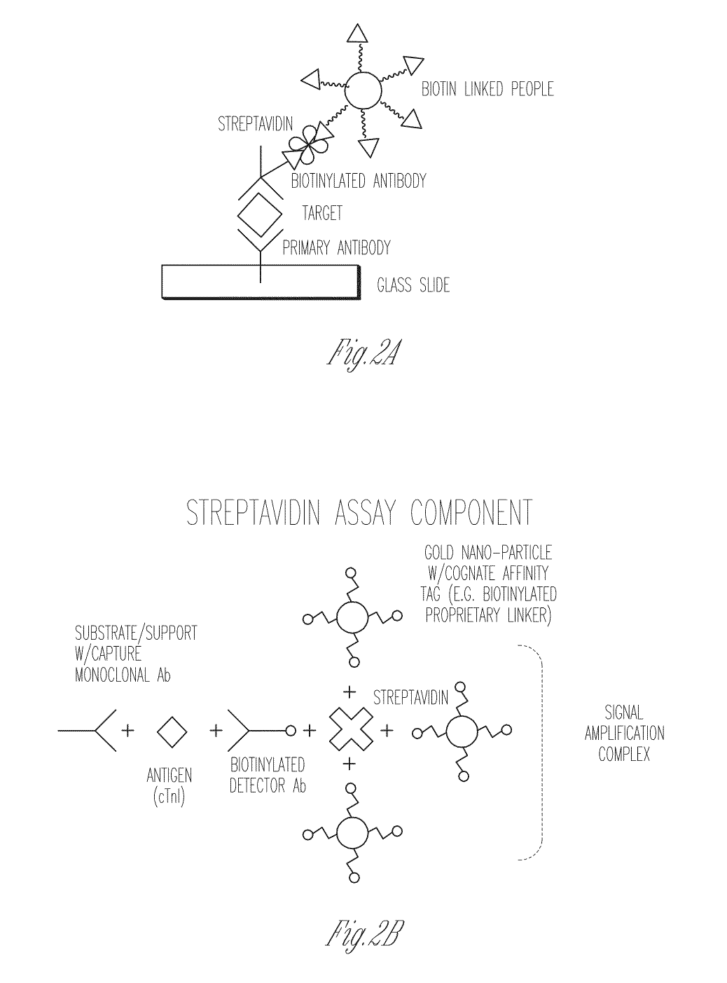

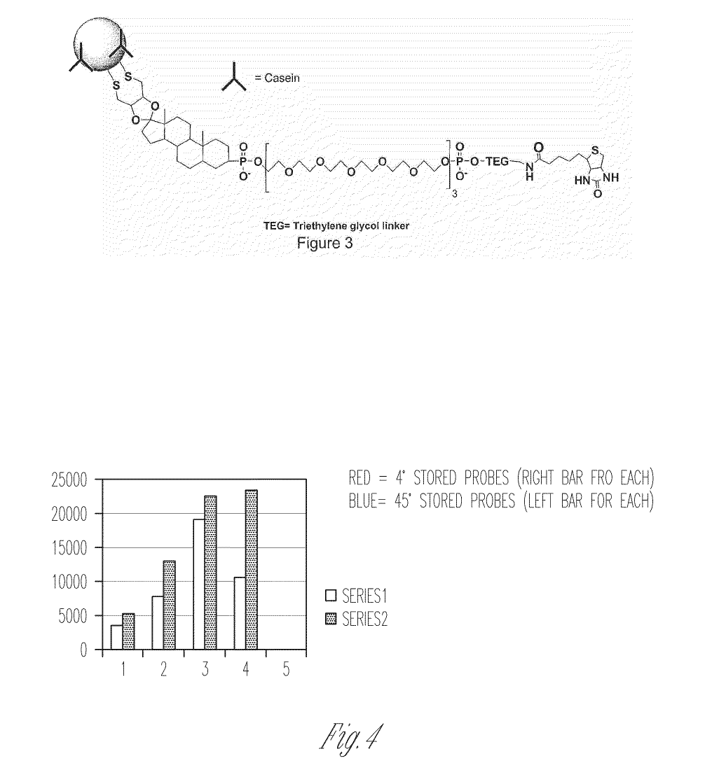

In one embodiment, the invention described herein provides a simple method of making stable, disulfide linked PEG-biotin modified gold nanoparticles and a method of target detection using Epi-disulfide linked probes. 5′-Epi-(PEG)-3-biotin-3′ oligomers were synthesized with and without DNA base T and loaded on gold nanoparticles. These oligomers were loaded on 15 nm gold nanoparticles in different combinations and tested in target detection using buffer and serum conditions. A schematic diagram of target detection is shown in FIGS. 2A-B.

In one embodiment, an oligomer of the invention generally has formula (I): L-PO₂—O((-T-O—PO₂—O)ₘ—Zₙ)_{q}-biotin, wherein L represents a steroid with a cyclic disulfide functional group; T may be thymidine or any nucleobase, e.g., any nucleoside, or non-nucleoside compound, e.g., one that stabilizes a gold surface; each Z independently represents a polyglycol which may be the same or different; wherein n ranges from 3 to 20; wherein q ranges from 1 to 20; wherein m is 0 or 1; and wherein each polyglycol independently comprises from 1 to 60 carbon atoms and includes from 1 to 30 non-peroxide —O—. In one embodiment, an oligomer of the invention has formula (II): L-PO₂—O((-T-O—PO₂—O)ₘ—Zₙ)_{q}-biotin, wherein L represents a steroid with a cyclic disulfide functional group; T may be thymidine or any nucleobase, e.g., any nucleoside, or non-nucleoside compound, e.g., one that stabilizes a gold surface; each Z independently represents a polyethylene or triethylene glycol which may be the same or different, or a combination thereof; wherein n ranges from 3 to 20; wherein q ranges from 1 to 20; wherein m is 0 or 1; and wherein each Z independently comprises from 2 to 60 carbon atoms and includes from 1 to 30 non-peroxide —O—. In one embodiment, the oligomer of the invention has formula (III): L-PO₂—O((-T-O—PO₂—O)ₘ—Zₙ)_{q}-biotin, wherein L represents a steroid with a cyclic disulfide functional group; T represents thymidine but may be any nucleobase, e.g., any nucleoside, or non-nucleoside compound, e.g., that stabilizes a gold surface; each Z independently represents an aliphatic, aromatic, cyclic, acyclic, anionic or cationic linker having no more than 500 atoms, which may be the same or different; wherein n ranges from 3 to 20; wherein q ranges from 1 to 20; wherein m is 0 or 1; and wherein the linker does not substantially interfere with target binding or result in significant non-specific binding to surfaces, wherein Z is polyethylene glycol, e.g., diethylene glycol, triethylene glycol, tetraethylene glycol, pentaethylene glycol or hexaethylene glycol, —C₁-C₁₀-alkyl-, —COO—, —CH₂(CH₂)ᵥCOO—, —OCO—, R¹N(CH₂)ᵥ, —O—C(CH₂)ᵥ—, —O—(CH₂)ᵥ—O, wherein v is 0-30 and R¹ is H or is G(CH2)v, wherein G is —CH₃, —CHCH₃, —COOH, —CO₂(CH₂)ᵥCH₃, —OH, or —CH₂OH.

Nanoparticles useful in the practice of the invention include metal (e.g., gold, silver, copper and platinum), metal oxides (e.g., TiO₂), semiconductor (e.g., CdSe, CdS, and CdS or CdSe coated with ZnS) and magnetic (e.g., ferromagnetite) colloidal materials. Other nanoparticles useful in the practice of the invention include ZnS, ZnO, TiO₂, AgI, AgBr, HgI₂, PbS, PbSe, ZnTe, CdTe, InS₃, In₂Se₃, Cd₃Pb₂, Cd₃As₂, InAs, and GaAs. The size of the nanoparticles may be from about 5 nm to about 150 nm (mean diameter), e.g., from about 5 to about 50 nm or from about 10 to about 30 nm. The nanoparticles may also be rods.

Methods of making metal, semiconductor and magnetic nanoparticles are well-known in the art. Suitable nanoparticles are also commercially available from, e.g., Ted Pella, Inc. (gold), Amersham Biosciences (gold) and Nanoprobes, Inc. (gold). In one embodiment, the gold nanoparticles are those described in PCT/US01/01190, and U.S. Pat. No. 6,506,564, which are both incorporated herein by reference in their entirety. Gold colloidal particles may have high extinction coefficients.

To bind the probes to the nanoparticles, the probes are contacted with the nanoparticles in water for a time sufficient to allow at least some of the probe molecules to bind to the nanoparticles by means of the functional groups. Next, at least one salt is added to the water to form a salt solution. The salt can be any suitable water-soluble salt. For instance, the salt may be sodium chloride, lithium chloride, potassium chloride, cesium chloride, ammonium chloride, sodium nitrate, lithium nitrate, cesium nitrate, sodium acetate, lithium acetate, cesium acetate, ammonium acetate, a combination of two or more of these salts, or one of these salts in phosphate buffer. The salt can be added to the water all at one time or the salt is added gradually over time.

After adding the salt, the probes and nanoparticles are incubated in the salt solution for an additional period of time sufficient to allow sufficient additional polymer conjugates to bind to the nanoparticles to produce the stable nanoparticle-probes. The time of this incubation can be determined empirically. A total incubation time may be about 24-48 hours. The solution is then centrifuged and the nanoparticle probes processed as desired.

Nanoparticles probes of the invention have a variety of uses. For instance, they can be used as probes to detect or quantitate analytes. Analytes that may be detected or quantitated include but are not limited to polysaccharides, lipids, lipopolysaccharides, proteins, glycoproteins, lipoproteins, nucleoproteins, peptides, oligonucleotides, and nucleic acids. Specific analytes include antibodies, immunoglobulins, albumin, hemoglobin, coagulation factors, peptide and protein hormones (e.g., insulin, gonadotropin, somatotropin), non-peptide hormones, interleukins, interferons, other cytokines, peptides comprising a tumor-specific epitope (i.e., an epitope found only on a tumor-specific protein), cells (e.g., red blood cells), cell surface molecules (e.g., CD antigens, integrins, cell receptors), microorganisms (viruses, bacteria, parasites, molds, and fungi), fragments, portions, components or products of microorganisms, small organic molecules (e.g., digoxin, heroin, cocaine, morphine, mescaline, lysergic acid, tetrahydrocannabinol, cannabinol, steroids, pentamidine, and biotin), etc. Nucleic acids and oligonucleotides that can be detected or quantitated include genes (e.g., a gene associated with a particular disease), viral RNA and DNA, bacterial DNA, fungal DNA, mammalian DNA (e.g., human DNA), cDNA, mRNA, RNA and DNA fragments, oligonucleotides, synthetic oligonucleotides, modified oligonucleotides, single-stranded and double-stranded nucleic acids, natural and synthetic nucleic acids, and the like.

The analyte to be detected or a ligand therefore may be attached to a substrate. Suitable substrates include transparent solid surfaces (e.g., glass, such as a glass slide, quartz, plastics (e.g., wells of microtiter plates) and other polymers), opaque solid surface (e.g., white solid surfaces, such as TLC silica plates, filter paper, glass fiber filters, cellulose nitrate membranes, nylon membranes), and conducting solid surfaces (e.g., indium-tin-oxide (ITO)). The substrate can be any shape or thickness, but generally will be flat and thin. For instance, the substrate may be a glass slide and an antibody specific for the analyte may be attached to the substrate (see FIG. 2).

The invention will be further described by the following non-limiting examples.

### Example 1

An oligomer (3′-biotin-PEG-PEG-PEG-epiendrosterone disulfide-5′) was synthesized using 3′-biotin TEG CPG from Glen Research (cat no: 20-2955-10) with spacer phosphoramidites (cat no: 10-1918-02) and epiendrosterone phosphoramidite on oligo pilot at a 10 μmole scale. After synthesis was completed, the oligomer was deprotected using ammonia conditions at 55° C. for 2 hours, and then aliquoted into four tubes and evaporated to dryness. The oligomer was not purified on HPLC since it does not have a chromophore in the chain. The oligomer was quantitated on UV-Vis at 300 nm and, based on those readings, OD units were calculated prior to column loading.

An exemplary oligomer is shown below:

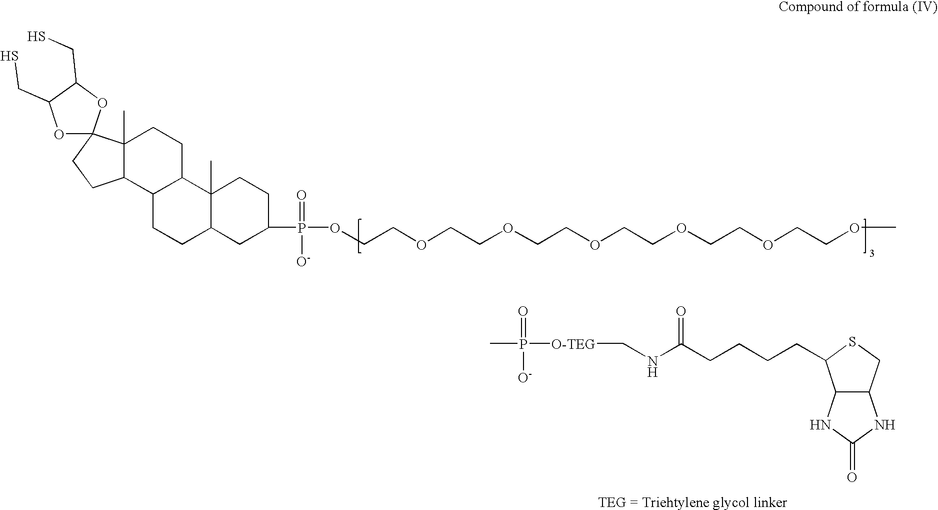

In place of PEG or TEG linkers, other linkers also can be used, for example, aliphatic, aromatic, cyclic and acyclic, anionic or cationic linkers and the like, so long as these linkers do not substantially interfere with target binding and do not result in substantial non-specific binding to the surface of substrates.

### Example 2

An oligomer (3′-biotin-PEG-T-PEG-T-PEG-T-epiendrosterone disulfide-5′) was synthesized using 3′-biotin TEG CPG from Glen Research (cat no: 20-2955-10) with spacer phosphoramidites (cat no: 10-1918-02), dT-CE phosphoramidite and epiendrosterone phosphoramidite on oligo pilot at a 10 μmole scale. After synthesis was completed, the oligomer was deprotected from the support using ammonia conditions at 55° C. for 2 hours. Then it was aliquoted into four tubes and evaporated to dryness. This oligomer was purified on HPLC since it has three “T” bases incorporated in the oligomer chain. After pooling the appropriate fractions, it was determined that the pool was 95.7% pure.

### Example 3

To prepare PEG-thiol gold colloids, 5 mL of 13 nM gold citrate was added to 5 mL of DNA grade water, yielding 6.5 nM gold citrate. To 10 mL of 6.5 nM gold citrate, 100 μL of 1% Tween 20 and 60 μL of 0.2% NaOH were added to make gold citrate pH 7. To this solution, 2 μM/mL oligomer (3′-biotin-PEG-PEG-PEG-Epi disulfide-5′) was added slowly and the reaction mixture was placed on a shaker at room temperature (Shaker RPM: 450). After 23 hours, an amount of casein solution was added to block the unreacted gold surface and shaking was continued for 1 hour (casein concentrations used in the preparations were 0.01%, 0.05% or 0.1%), after which the solution was aliquoted into 1.5 mL low retention tubes. All the aliquots were diluted with 500 μL of 0.01% Tween 20 solution and centrifuged for 25 minutes at 12,000 RCF. The supernatants were removed and the pellets resuspended in 0.01% Tween 20 solution and then subjected to centrifugation for 25 minutes at 12,000 RCF. The supernatants were removed from the tubes and the pellets were dissolved in 1×PBS, 0.1% BSA, 0.05% Tween 20 buffer. Probes were quantitated using 2 μL of solution. A yield of 92% was obtained in presence of casein and a yield of 62% was obtained without casein blocking.

Following the same method, 3′-PEG-T-PEG-T-PEG-T-Epi disulfide probes, and probes having 3′-TGTGC-5H-5′ DNA filler to control the orientation of the probes, were prepared. However, DNA filler addition did not increase the sensitivity of the probes.

### Example 4

After preparing probes using different concentrations of casein blocking, the probes were tested in a simple assay format using biotin PEG printed slides and streptavidin as a target. The slides were washed with 1×PBS buffer, 0.3% Tween 20 and then blocked with 1×PBS, 1% BSA and 0.3% Tween 20. The blocked slides were treated with 100 μL streptavidin (20 ng/100 μL) in binding buffer for 10 minutes at 35° C., washed, and then treated with 500 μM probe in binding buffer. After washing, the signal was amplified with silver for 7 minutes.

### Results

As shown in FIG. 4, as casein concentration in the blocking step increased, signal intensity in the assay also increased, particularly for low concentration of captures on the surface. Probes stored at 4° C. were compared with probes stored at 45° C. for 48 hours. After 48 hours at 45° C. probes

### Example 5

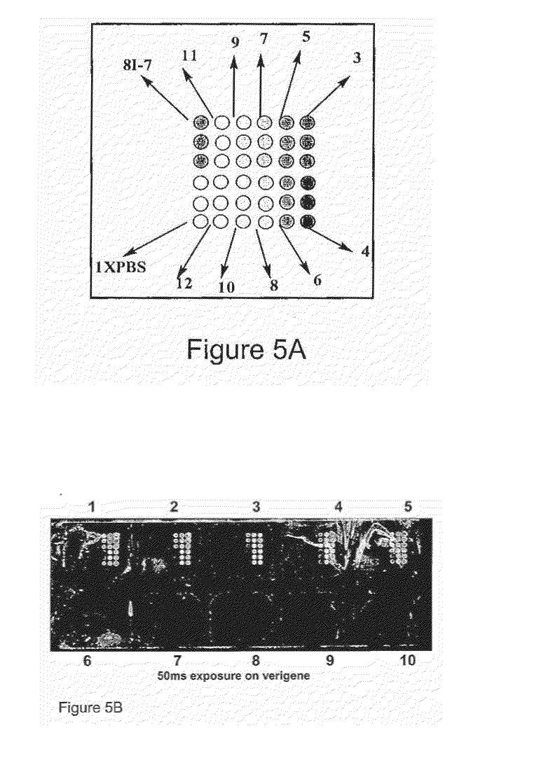

Probes were used in a streptavidin binding experiment in 50% serum. After blocking the slide, streptavidin bound to the captures (which may be an antibody linked to strepavidin or a compound of the invention amine linked to a support, e.g., via N-hydroxy succinamide) was treated with 500 μM probe at 35° C. (FIG. 5). This experiment was performed using Codelink surface.

### Results

All the probes worked well in normal serum at 35° C. Signal was observed starting at 100 pg/μL capture in all the wells. No capping and BSA capping probes showed very slight background at 1000 ms exposure.

### Example 6

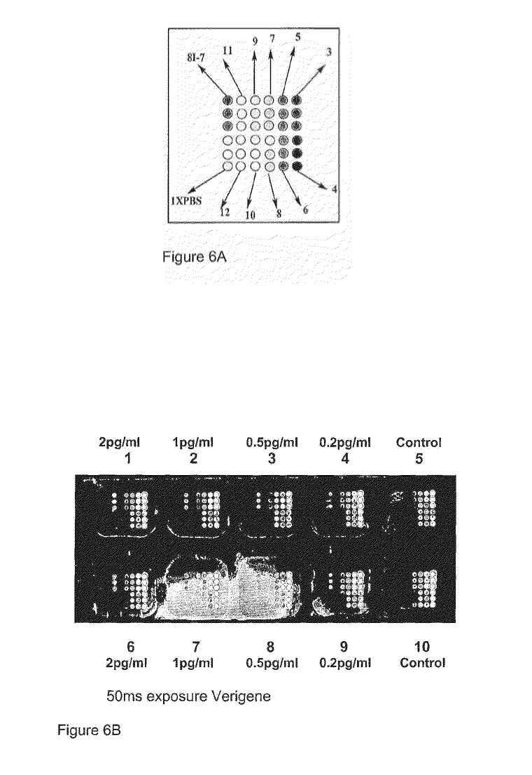

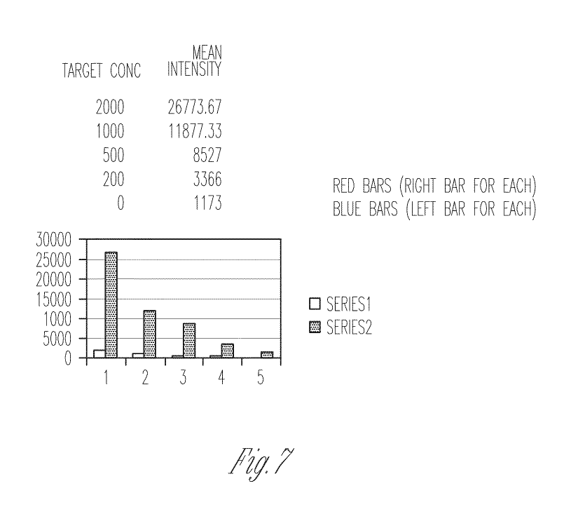

The samples were in depleted serum at 35° C. using a troponin target (FIG. 6). The probe was a 5′-Peg-Peg-Peg-biotin 3′ oligomer and 0.1% casein capping was employed. Target was added for 1 hour, secondary antibody was added for 30 minutes, streptavidin was added for 10 minutes, probe was added for 10 minutes and silver staining was conducted for 9 minutes. In this experiment, a dose response was observed from 20 pg/mL to 0.2 pg/mL. Target could be detected at 0.2 pg/ml (Table 1 and FIG. 7).

TABLE 1

| | Mean |
|---|---|
| Target conc | Intensity |
| 2000 | 26773.67 |
| 1000 | 11877.33 |
| 500 | 8527 |
| 200 | 3366 |
| 0 | 1173 |

### Example 7

To determine probe stability at 45° C., four different types of casein blocked peg thiol probes were used. It was observed that all probes were still active in a sandwich assay after 22 days at 45° C. in a cyclo-olefin copolymer (COC) bottle. Among the tested probes, 0.05% and 0.1% casein blocked probes showed higher signals compared to 0.01% casein blocked probe and probe without casein blocking. In a typical experiment, 500 pM peg thiol probes were incubated at 45° C. in COC bottles for 22 days in 1×PBS, 0.3% Tween 20, 1% BSA. Then probe aliquots were diluted 10 times before using in the assay. The final concentration of the probe used in the assay format was 50 pM.

A printed codelink slide was blocked for 1 hour with blocking buffer (1×PBS-1% BSA-0.3% Tween 20), then treated with 100 μL of streptavidin (200 ng/mL) in binding buffer (1×PBS-1% BSA-0.3% Tween 20) for 10 minutes for each well on the slide. Next, each well was treated with 50 pM probe for 10 minutes. Finally, all the wells were treated with silver A and B (Immunopure from Pierce, Cat no: 21125, silver solutions A: E70-0074D007; and B6: E70-0251D001) for 6 minutes.

TABLE 2

| Well | | Probe stored | | |
|---|---|---|---|---|
| no | Probe used | temperature | Target used | Conclusions |
| 1 | 5′-epi-peg-peg-peg | 4° C. | Streptavidin | Probe efficiently detects |
| | biotin-3′ | | | until 200 pg/μL capture |
| 2 | 0.01% casein (C: 5679 | 4° C. | Streptavidin | Probe efficiently detects |
| | Sigma Aldrich) blocked | | | until 500 pg/μL capture |
| | probe with peg biotin | | | |
| | oligomer | | | |
| 3 | 0.05% casein (C: 5679 | 4° C. | Streptavidin | Probe efficiently detects |
| | Sigma Aldrich) blocked | | | until 500 pg/μL capture |
| | probe with peg biotin | | | |
| | oligomer | | | |
| 4 | 0.1% casein (C: 5679 | 4° C. | Streptavidin | Probe efficiently detects |
| | Sigma Aldrich) blocked | | | until 500 pg/μL capture |
| | probe with peg biotin | | | |
| | oligomer | | | |
| 5 | No probe | — | Streptavidin | No silver amplification on |
| | | | | streptavidin and only |
| | | | | general background from |
| | | | | silver |
| 6 | 5′-epi-peg-peg-peg- | 45° C. | Streptavidin | Probe efficiently detects 5 |
| | biotin-3′ (50 pM) | | | and 2 μg/μL captures |
| 7 | 0.01% casein (C: 5679 | 45° C. | Streptavidin | Probe efficiently detects 5 |
| | Sigma Aldrich) blocked | | | and 2 μg/μL captures |
| | probe with peg biotin | | | |
| | oligomer (50 pM) | | | |
| 8 | 0.05% casein (C: 5679 | 45° C. | Streptavidin | Probe efficiently detects 5 |
| | Sigma Aldrich) blocked | | | and 2 μg/μL captures |
| | probe with peg biotin | | | |
| | ologimer (50 pM) | | | |
| 9 | 0.1% casein (C: 5679 | 45° C. | Streptavidin | Probe efficiently detects 5 |
| | Sigma Aldrich) blocked | | | and 2 μg/μL captures |
| | probe with peg biotin | | | |
| | oligomer (50 pM) | | | |
| 10 | No probe | — | Streptavidin | No silver amplification on |
| | | | | streptavidin and only |
| | | | | general background from |
| | | | | silver |

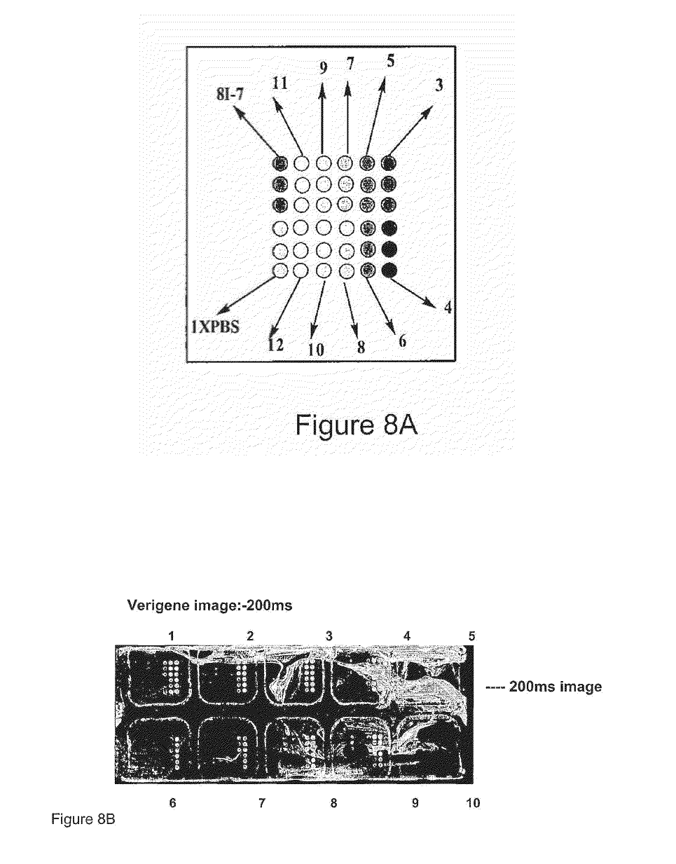

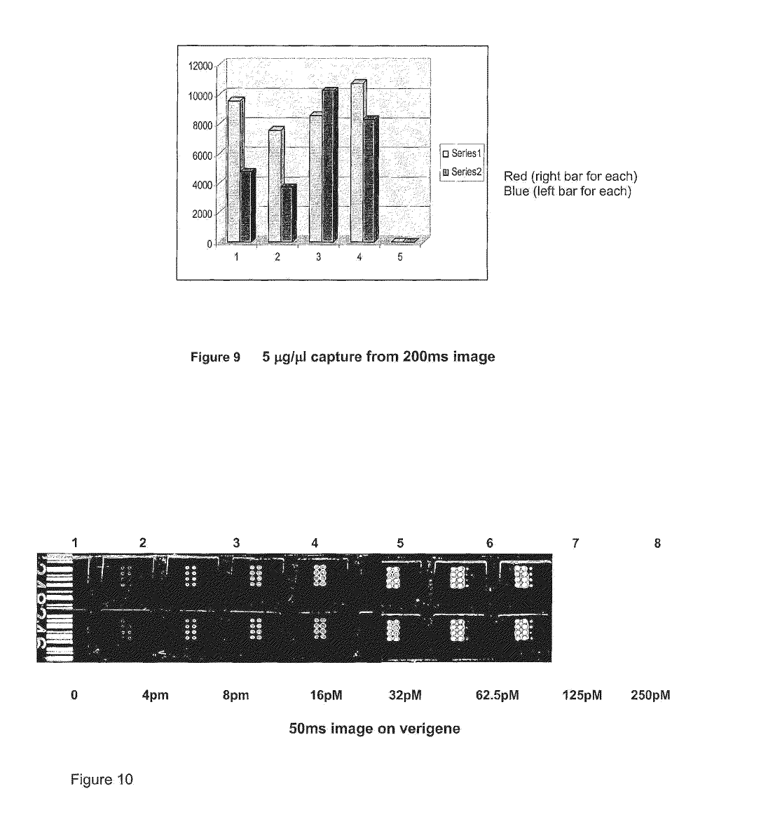

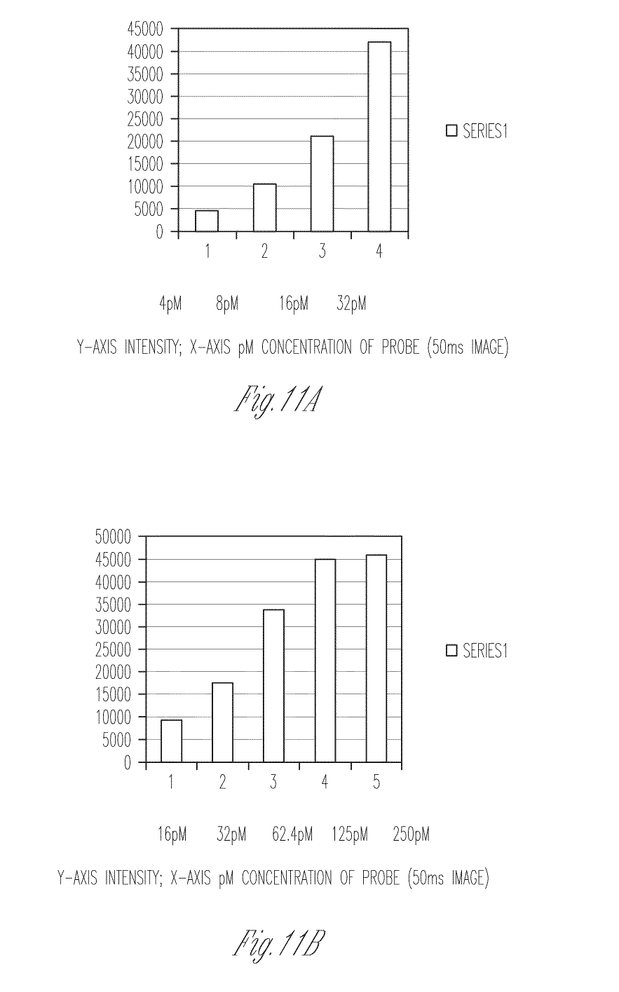

Different concentrations of the peg thiol captures were printed on the slide and quantitated (5 μg/μL capture from 200 ms image; FIG. 8).

0.05% and 0.1% casein blocked probes were active after storing in COC bottles for 22 days at 45° C.

### Example 8

Hydrogel slides were printed using goat anti-mouse antibody (200 μg/mL) and 3′ amine linked peg-biotin (1 mg/mL) captures in 8×2 matrix. These slides were hydrated overnight after printing and used after that.

For the assay, a printed slide was blocked with blocking buffer (50 mM Tris pH 8; 50 mM NaCl; 0.01% Tween 20 and 50 mm ethanolamine) for 60 minutes. Then 100 μL of 100 ng/mL streptavidin in binding buffer (1×PBS, 1% BSA and 0.3% Tween 20) was added to each well and kept at room temperature for 10 minutes. Then each well was washed with wash buffer (1×PBS and 0.3% Tween 20) three times to remove the excess streptavidin from the wells. Different concentrations of peg-biotin probe were added to each well to complete the sandwich hybridization. After 10 minutes, each well was washed with wash buffer and treated with a silver A and B mixture for 6 minutes at room temperature. The slide was then washed thoroughly with filtered water and scanned on Verigene under different exposures (10 ms, 20 ms, 50 ms, 100 ms, 200 ms, 500 ms, 1000 ms).

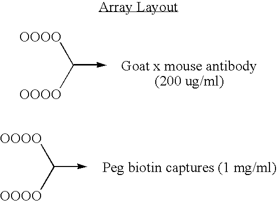

After imaging on Verigene, spots on the slide were quantitated using Genepix software for analysis. The probe gave very good dose response at low concentrations (from 4 pM to 62.5 pM) without any well background. This experiment shows that at 4 pM concentration, the probe is detectable in a streptavidin based sandwich assay (FIGS. 11A-B).

### Example 9

5′-Epi disulfide-(PEG)-3-biotin-3′ labeled gold nanoparticles were prepared using casein as a blocking agent (24 hours at room temperature). 5′-Epi disulfide-(PEG)-3-biotin-3′ oligomers were prepared using commercially available PEG phosphoramidite and a disulfide linker epi-disulfide-phosphoramidite on a gene synthesizer. Oligomers were prepared with and without nucleotide “T” (deoxyribothymidine phosphate) between PEG linkers. The presence of nucleotide T allows for easy quantitation and purification. Probes were loaded using both oligomers and compared side by side in target detection. Oligomers ranging from 1 μM to 8 μM were loaded on 1:1 diluted 15 nm gold nanoparticles (6.4 nM concentration). After comparing all the preparations, it was determined that 2 μM loading probe resulted in an improved yield and the absence of aggregation during preparation and workup. The use of higher concentrations, e.g., 4 μM and 8 μM, resulted in non-specific binding to surfaces. Longer incubation (>48 hours) also resulted in aggregation and non-specific binding to glass surfaces.

In summary, surprisingly, casein blocking improved detection at low captured target concentrations. In particular, as the concentration of casein as a blocking agent was increased, signal intensity was increased at a low concentration of captures on the slide without increasing the background of the control spots. These probes were used in buffer as well in serum conditions for target detection. In both the cases, probes showed specificity and sensitivity (e.g., at 0.2 pg/mL) in troponin target detection on Code link and hydrogel surfaces. Thus, these probes can be used in ultra sensitive target detection (proteins, DNA and the like) using streptavidin as a glutinating agent for detection.

### REFERENCES

Synthesis and Grafting of Thioctic Acid-PEG-Folate Conjugates onto Au Nanoparticles for Selective Targeting of Folate Receptor-Positive Tumor Cells, Bioconjugate Chem., 17 (3), 603-609, 2006, Vivechana Dixit, Jeroen Van den Bossche, Debra M. Sherman, David H. Thompson, and Ronald P. Andres.
Enhanced oligonucleotide-nanoparticle conjugate stability using thioctic acid modified oligonucleotides.; Dougan J A, Karlsson C, Smith W E, Graham D, Nucleic Acids Res. 2007 May 8.
Functionalization of thioctic acid-capped gold nanoparticles for specific immobilization of histidine-tagged proteins, Abad J M, Mertens S F, Pita M, Fernández V M, Schiffrin D J.; J Am Chem. Soc. 2005 Apr. 20; 127(15):5689-94.
Thioctic acid amides: convenient tethers for achieving low nonspecific protein binding to carbohydrates presented on gold surfaces, Chem. Commun., 2005, 3334-3336, DOI: 10.1039/b503843j, Rositsa Karamanska, Balaram Mukhopadhyay, David A. Russell and Robert A. Field.
U.S. Pat. No. 7,253,277, and references cited there.
Use of a Steroid Cyclic Disulfide Anchor in Constructing Gold Nanoparticle-Oligonucleotide Conjugates; R. L. Letsinger, R. Elghanian, G. Viswanadham, and C. A. Mirkin; pp 289-291.
Colloidal Gold Nanoparticles: A Versatile Platform for Developing Tumor Targeted Cancer Therapies; May 5, 2005 By: G. F. Paciotti et al., CytImmune Sciences, Inc.
Colloidal gold nanoparticles: a novel nanoparticle platform for developing multifunctional tumor-targeted drug delivery vectors (p 47-54); Giulio F. Paciotti, David G. I. Kingston, Lawrence Tamarkin; Published Online: May 11, 2006 4:23 PM; Drug Development Research.
PEG-modified gold nanorods with a stealth character for in vivo applications; Takuro Niidome, Masato Yamagata, Yuri Okamoto, Yasuyuki Akiyama, Hironobu Takahashi, Takahito Kawano, Yoshiki Katayama and Yasuro Niidome; Journal of Controlled Release; Volume 114, issue 3; pp 343-347.

All publications, patents and patent applications are incorporated herein by reference. While in the foregoing specification, this invention has been described in relation to certain embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A compound of formula (I): L-PO₂—O((-T-O—PO₂—O)ₘ—Zₙ)_{q}-biotin, wherein L represents a steroid with a cyclic disulfide functional group; wherein T represents thymidine; wherein each Z independently represents a polyglycol which may be the same or different; wherein n ranges from 3 to 20; wherein q ranges from 1 to 20; wherein m is 0 or 1; and wherein each polyglycol independently comprises from 1 to 60 carbon atoms and includes from 1 to 30 non-peroxide —O—.

2. The compound of claim 1 wherein n is 4.

3. The compound of claim 1 wherein m is 0.

4. The compound of claim 1 wherein m is 0, q is 1, n is 4, and Z is (C₁₂O₆)₃ or C₆O₃ or any combination thereof.

5. The compound of claim 1 wherein m is 1, Z is (C₁₂O₆), n is 1, and q is 3.

6. The compound of claim 1 wherein Z is a polyethylene.

7. A preparation of gold nanoparticles coupled to the compound of claim 1.

8. The preparation of claim 7 further comprising an amount of a protein that stabilizes the preparation, reduces non-specific binding, reduces agglomeration, or any combination thereof.

9. The preparation of claim 8 wherein the protein comprises casein.

10. The preparation of claim 8 wherein the protein comprises albumin.

11. A kit having the compound of claim 1.

12. A kit having the preparation of claim 7.

13. The kit of claim 12 further comprising an amount of a protein that stabilizes the preparation, reduces non-specific binding, reduces agglomeration, or any combination thereof.

14. The kit of claim 13 wherein the protein comprises casein.

15. The kit of claim 13 wherein the protein comprises albumin.

16. A method of detecting the presence or amount an analyte in a sample, comprising:
a) providing a support having a ligand specific for the analyte attached thereto;
b) contacting the support with a sample suspected of having the analyte so as to yield a mixture;
c) contacting the mixture with an amount of the preparation of claim 7, an avidin labeled molecule, and a biotinylated ligand specific for the analyte; and
d) detecting the presence or amount of the gold nanoparticles bound to the support, thereby detecting the presence or amount of the analyte in the sample.

17. The method of claim 16 wherein the avidin labeled molecule comprises streptavidin.

18. The method of claim 16 wherein the ligand specific for the analyte comprises an antibody or antigen binding fragment thereof.

19. The method of claim 16 wherein biotinylated ligand comprises an antibody or antigen binding fragment thereof.

20. The method of claim 16 wherein at least 0.2 pg/mL of the analyte can be detected.

21. The method of claim 16 wherein the amount of preparation is prepared by contacting the nanoparticles with about 0.1 to about 10 μM of the compound.
